(19) United States
(12) Patent Application Publication
(10) Pub. No.: US 2009/0317367 A1
(43) Pub. Date: Dec. 24, 2009

(54) METHODS OF PRODUCING PREADIPOCYTES AND INCREASING THE PROLIFERATION OF ADULT ADIPOSE STEM/PROGENITOR CELLS

(75) Inventors: Gregorio Chazenbalk, Studio City, CA (US); Cristina Bertolotto, Los Angeles, CA (US); Ricardo Azziz, Beverly Hills, CA (US); Charles F. Simmons, Jr., Los Angeles, CA (US); Saleh Heneidi, Long Beach, CA (US)

Correspondence Address: DAVIS WRIGHT TREMAINE LLP/Los Angeles, 865 FIGUEROA STREET, SUITE 2400, LOS ANGELES, CA, 90017-2566, US

(73) Assignee: CEDARS-SINAI MEDICAL CENTER, Los Angeles, CA (US)

(21) Appl. No.: 12/528,286

(22) PCT Filed: Feb. 21, 2008
(86) PCT No.: PCT/US08/54548

§ 371 Date: Aug. 21, 2009

Related U.S. Application Data
(60) Provisional application No. 60/891,022, filed on Feb. 21, 2007.

(51) Int. Cl. A61K 35/12; C12N 5/08; C12N 5/02; A61P 17/00; A61P 9/00; A61P 25/00; A61P 19/00; A61P 31/12

(52) U.S. Cl. 424/93.7; 435/373; 435/366; 435/377

(57) ABSTRACT

The present invention describes preadipocytes and methods of differentiating macrophages into preadipocytes by co-culturing adipocytes and resident adipose tissue macrophages. Also described are methods of increasing the proliferative rate of adipose adult stem/progenitor cells.

### FIELD OF INVENTION

This invention relates to preadipocytes and adipose stem and progenitor cells.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Obesity has reached epidemic proportions with more than 1 billion adults being overweight and at least 300 million of those clinically obese. Primarily the result of defects in energy balance (Gimeno, R. E. and L. D. Klaman, Adipose tissue as an active endocrine organ: recent advances. Curr Opin Pharmacol, 2005. 5(2): p. 122-8; Reaven, G. M., The metabolic syndrome: requiescat in pace. Clin Chem, 2005. 51(6): p. 931-8), obesity is a major contributor to chronic disease and disability, including type 2 diabetes mellitus (DM), cardiovascular disease, infertility, gallbladder disease, sleep apnea, chronic musculoskeletal problems such as osteoarthritis, depression, and certain forms of cancer, especially hormonally related and large-bowel cancers (Bouloumie et al., Role of macrophage tissue infiltration in metabolic diseases. Curr Opin Clin Nutr Metab Care, 2005. 8(4): p. 347-54). The role of adipose tissue in obesity was thought to have been a passive one, however, today it is understood that adipocytes play a much more active role in metabolism, including interacting with the immune system through inflammatory mediators and signaling molecules (Greenberg, A. S. and M. S. Obin, Obesity and the role of adipose tissue in inflammation and metabolism. Am J Clin Nutr, 2006. 83(2): p. 461S-465S; Fantuzzi, G., Adipose tissue, adipokines, and inflammation. J Allergy Clin Immunol, 2005. 115(5): p. 911-9; quiz 920). This inflammatory response appears to be critical in the development of obesity and its later sequelae, of insulin resistance.

Several lines of evidence demonstrate that obesity is a chronic low-grade inflammatory state. Increased obesity is associated with accumulation of macrophages in adipose tissue, which are attracted to adipose tissue as the degree of obesity worsens (Odegaard et al., Macrophage-specific PPARgamma controls alternative activation and improves insulin resistance. Nature, 2007. 447(7148): p. 1116-20). The increased recruitment of macrophages and increased production of cytokines and adipokines by both macrophages and adipocytes may result from cellular stress and death due to hypoxia, hyperglycemia, or a number of other metabolic stressors (Chinetti et al., Expression of adiponectin receptors in human macrophages and regulation by agonists of the nuclear receptors PPARalpha, PPARgamma, and LXR. Biochem Biophys Res Commun, 2004. 314(1): p. 151-8). Macrophages influence adipocyte growth, metabolism and secretory activity through the production of cytokines and chemokines (Xu et al., Chronic inflammation in fat plays a crucial role in the development of obesity-related insulin resistance. J Clin Invest, 2003. 112(12): p. 1821-30; Suganami et al., A paracrine loop between adipocytes and macrophages aggravates inflammatory changes: role of free fatty acids and tumor necrosis factor alpha. Arterioscler Thromb Vasc Biol, 2005. 25(10): p. 2062-8). In turn, adipocytes may also modulate macrophage function since macrophages express receptors to both leptin and adiponectin (Gainsford et al., Leptin can induce proliferation, differentiation, and functional activation of hemopoietic cells. Proc Natl Acad Sci USA, 1996. 93(25): p. 14564-8; Weisberg et al., Obesity is associated with macrophage accumulation in adipose tissue. J Clin Invest, 2003. 112(12): p. 1796-808). In vitro co-culture of differentiated 3T3-L1 adipocytes and RAW 264 macrophages results in significant upregulation of proinflammatory cytokines and downregulation of anti-inflammatory cytokines (Suganami et al., A paracrine loop between adipocytes and macrophages aggravates inflammatory changes: role of free fatty acids and tumor necrosis factor alpha. Arterioscler Thromb Vasc Biol, 2005. 25(10): p. 2062-8). Furthermore, the interaction of 3T3-L1 adipocytes with mouse peritoneal macrophages mediates the production of factors from macrophages that influence insulin sensitivity in adipocytes (Lumeng et al., Macrophages block insulin action in adipocytes by altering expression of signaling and glucose transport proteins. Am J Physiol Endocrinol Metab, 2007. 292(1): p. E166-74). Understanding the interaction between adipocytes and macrophages may elucidate mechanisms underlying the etiology of excess adiposity and obesity.

Adipose tissue is not only composed of adipocytes, macrophages, and vascular tissue, but also contains adult adipose stem/progenitor cells (AASPCs) located in the stroma vascular fraction (Zuk et al., Human adipose tissue is a source of multipotent stem cells. Mol Biol Cell, 2002. 13(12): p. 4279-95; Fraser et al., Fat tissue: an underappreciated source of stem cells for biotechnology. Trends Biotechnol, 2006. 24(4): p. 150-4). AASPCs can differentiate along adipocyte, osteoblast, chondrocyte, and other mesenchymal pathways in a manner similar to that of multipotent stromal cells derived from bone marrow, demonstrating a potentially high degree of plasticity in these cells (Fraser et al., Fat tissue: an underappreciated source of stem cells for biotechnology. Trends Biotechnol, 2006. 24(4): p. 150-4; Moseley et al., Adipose-derived stem and progenitor cells as fillers in plastic and reconstructive surgery. Plast Reconstr Surg, 2006. 118(3 Suppl): p. 121S-128S; Charriere, G., et al., Preadipocyte conversion to macrophage. Evidence of plasticity. J Biol Chem, 2003. 278(11): p. 9850-5). Mature adipocytes do not exhibit proliferative activity, so any increase in adipocyte numbers is due to differentiation of the immediate adipocyte precursors, the preadipocytes. While a clear distinction has yet to be drawn, it appears that multiple populations of preadipocytes exist, with varied origins, and only a fraction of these preadipocytes are true AASPCs (Zuk et al., Human adipose tissue is a source of multipotent stem cells. Mol Biol Cell, 2002. 13(12): p. 4279-95; Fraser et al., Fat tissue: an underappreciated source of stem cells for biotechnology. Trends Biotechnol, 2006. 24(4): p. 150-4; Moseley et al., Adipose-derived stem and progenitor cells as fillers in plastic and reconstructive surgery. Plast Reconstr Surg, 2006. 118(3 Suppl): p. 121S-128S; Charriere et al., Preadipocyte conversion to macrophage. Evidence of plasticity. J Biol Chem, 2003. 278(11): p. 9850-5). Preadipocytes differentiate to adipocytes (Charriere et al., Preadipocyte conversion to macrophage. Evidence of plasticity. J Biol Chem, 2003. 278(11): p. 9850-5; Devitt et al., Persistence of apoptotic cells without autoimmune disease or inflammation in CD14−/− mice. J Cell Biol, 2004. 167(6): p. 1161-70; Reichert, M. and D. Eick, Analysis of cell cycle arrest in adipocyte differentiation. Oncogene, 1999. 18(2): p. 459-66) and, in turn, adipocytes can dedifferentiate to preadipocytes (Yagi et al., A novel preadipocyte cell line established from mouse adult mature adipocytes. Biochem Biophys Res Commun, 2004. 321(4): p. 967-74). Recently, adipocyte precursors, preadipocytes, have also been observed to rapidly and efficiently differentiate into typical macrophages (Cousin et al., A role for preadipocytes as macrophage-like cells. Faseb J, 1999. 13(2): p. 305-12; Prunet-Marcassus et al., From heterogeneity to plasticity in adipose tissues: site-specific differences. Exp Cell Res, 2006. 312(6): p. 727-36). Even embryonic stem cells (ESCs) share some of the phagocytic characteristics of macrophages (Charriere et al., Macrophage characteristics of stem cells revealed by transcriptome profiling. Exp Cell Res, 2006. 312(17): p. 3205-14).

With the AASPCs' ability to differentiate along adipocyte, osteoblast, chondrocyte and other mesenchymal pathways, methods to increase the proliferation of AASPCs will be useful in tissue engineering and regenerative medicine. Thus, there is a need in the art for a method to increase the proliferation of AASPCs and a method to generate preadipocytes, which may transform into mature adipocytes.

Furthermore there is also a need in the art for the generation of adult stem/progenitor cells from a readily available tissue. Adipose tissue provides a readily available tissue source, and thus, provides another reason for methods to increase the proliferation of AASPCs.

### SUMMARY OF THE INVENTION

The following embodiments and aspects thereof are described and illustrated in conjunction with compositions and methods which are meant to be exemplary and illustrative, not limiting in scope.

The present invention describes a method of producing preadipocytes, comprising: providing a population of macrophages and a population of adipocytes; and co-culturing the population of macrophages and the population of adipocytes to differentiate at least one macrophage into a preadipocyte. The population of macrophages may be isolated from adipose tissue. The co-culturing may comprise co-culturing the population of macrophages and the population of adipocytes for about 24 hours. In a further embodiment, the method may further comprise separating the population of macrophages and the population of adipocytes and further culturing the population of macrophages.

The present invention also describes a population of preadipocytes produced by the these co-culturing methods.

The present invention additionally describes a method of increasing the proliferative rate of adult adipose stem/progenitor cells (“AASPCs”), comprising: providing a population of adipocytes and a population of macrophages comprising an AASPC; and co-culturing the population of adipocytes and the population of macrophages, whereby the proliferative rate of the AASPC increases. The population of macrophages may be isolated from adipose tissue. The co-culturing may comprise co-culturing the population of adipocytes and the population of macrophages comprising the AASPC for about 24 hours.

The present invention also describes a population of AASPCs produced by these co-culturing methods.

The present invention further describes a method of producing preadipocytes, comprising: providing a population of adult adipose stem/progenitor cells (“AASPCs”) produced by: providing a population of adipocytes and a population of macrophages comprising an AASPC, and co-culturing the population of adipocytes and the population of macrophages to increase the proliferative rate of the AASPC to produce the population of AASPCs; and differentiating the population of AASPCs into preadipocytes. The population of macrophages may be isolated from adipose tissue. The co-culturing may comprise co-culturing the population of adipocytes and the population of macrophages comprising the AASPC for about 24 hours.

The present invention also describes a method of using adult adipose stem/progenitor cells (“AASPCs”) to treat a condition in a subject in need thereof, comprising: providing a quantity of AASPCs; and administering a therapeutically effective amount of the quantity of AASPCs to the subject to treat the condition. The condition treated may include but is not limited to cardiac disease, neurological deficit, bone injury, cartilage injury, joint injury, degenerative spinal disease, vascular disease, viral infection, and tissue damage. In a particular embodiment, the condition is tissue damage and the quantity of AASPCs promotes tissue regeneration.

Other features and advantages of the invention will become apparent from the following detailed description, taken in conjunction with the accompanying drawings, which illustrate, by way of example, various features of embodiments of the invention.

### BRIEF DESCRIPTION OF THE FIGURES

Exemplary embodiments are illustrated in referenced figures. It is intended that the embodiments and figures disclosed herein are considered illustrative rather than restrictive.

FIG. 1 depicts immunofluorescence of S-100, CD14, and DAPI in adipose tissue in accordance with an embodiment of the present invention. CD14 expression displays points of interaction between adipocytes and resident macrophages in tissue as normally occurring in vivo cross-talk.

FIG. 2 depicts immunofluorescence of S-100, CD34, and DAPI in adipose tissue in accordance with an embodiment of the present invention. CD34 expression displays points of interaction between adipocytes and resident macrophages in tissue as normally occurring in vivo cross-talk.

FIG. 3 depicts mature adipocytes (A) and isolated resident macrophages (B) in accordance with an embodiment of the present invention. (A) Oil Red 0 (red) co-labeled with DAPI (light blue) clearly indicates mature adipocytes, with a magnification of 100×. (B) Isolated resident macrophages after 3 days in culture, labeled for CD14, magnification of 200×.

FIG. 4 depicts macrophage sections and adipocyte sections in accordance with an embodiment of the present invention. (A) Macrophage section left untreated for 3 days. (B) Adipocyte section after co-culture with macrophages for 24 hours; some adipocyte growth with moderate preadipocyte formation is observed. (C) Macrophage section after co-culture for 24 hours; preadipocytes have formed and reached complete confluency. (D) Comparison of adiponectin levels from macrophage section with and without co-culture.

FIG. 5 depicts immunofluorescent characterization of preadipocytes resulting from co-culture in accordance with an embodiment of the present invention. All images have a magnification of 200×.

FIG. 6 depicts CD14(+) nanobeads incorporated into cells of the macrophage fraction in accordance with an embodiment of the present invention. (A) Nanobeads in red are seen in the macrophage fraction before co-culture. (B) Nanobeads are seen in fully differentiated preadipocytes.

FIG. 7 depicts immunofluorescence diagram of the morphological changes exhibited during various stages of macrophage differentiation to preadipocytes and expression of key markers CD14 (green), S100 (red), and CD34 (results not shown) in accordance with an embodiment of the present invention. Macrophages (left) slowly begin to express S100, a protein found in preadipocytes but not macrophages, as they transform to preadipocytes (right), while maintaining CD14 expression.

FIG. 8 depicts AASPCs in accordance with an embodiment of the present invention. (A) Two preadipocytes, both CD34(+) (green), with top preadipocyte engulfed by AASPCs. (B) Preadipocyte phagocytized by AASPCs, bottom, with untouched preadipocyte, right. (C) AASPC is clearly DAPI positive (blue). (D) AASPC is S-100(+) (red) and CD34(+) (green).

FIG. 9 depicts human sphere progenitors derived after cross talk between adipocytes and macrophages in accordance with an embodiment of the present invention. Blue DAPI, Red S-100 (preadipocytes marker), and Green, CD34 (Hematopoietic and endothelial stem cell marker).

FIG. 10 depicts the process of the differentiation of adipocyte stem cells spheres to preadipocytes cells in accordance with an embodiment of the present invention. (A) Sphere with multiples small nucleus and is positive for CD34, S-100 and the nucleus are label with DAPI. (B) The sequence from figure one, the cells are bigger, and positive for CD-105, DLK and nucleus label with DAPI. (C) The cells are more mature emerging from the sphere, they are positive for CD-68, DLK and nucleus are label with DAPI. (D) The cells are label for CD-68 and DLK with the nucleus label with DAPI. This figure demonstrates that the cells' shape is fusiform and they are positive for typical preadipocytes markers.

FIG. 11 depicts preadipocytes derived from sphere stem cells in accordance with an embodiment of the present invention. Markers used to identify cells: CD 34: Hematopoietic progenitor cell antigen cell surface, label macrophages and monocytes; CD 68: recognize macrophages; S-100: a selective marker of preadipocytes and newly formed adipocytes; S-105: mesenchymal stem cell marker; DLK: preadipocytes factor marker.

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., J. Wiley & Sons (New York, N.Y. 2001); March, Advanced Organic Chemistry Reactions, Mechanisms and Structure 5^{th} ed., J. Wiley & Sons (New York, N.Y. 2001); and Sambrook and Russel, Molecular Cloning: A Laboratory Manual 3^{rd} ed., Cold Spring Harbor Laboratory Press (Cold Spring Harbor, N.Y. 2001), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

“Stem cell” as used herein refers to a cell that can continuously produce unaltered daughters and also has the ability to produce daughter cells that have different, more restricted properties.

“Adult stem cell” as used herein refers to a pluripotent or a multipotent stem cell from fetal or adult sources.

“Tissue stem cell” as used herein refers to a stem cell derived from, or resident in, a fetal or adult tissue, with potency limited to cells of that tissue.

“Progenitor cell” as used herein refers to a dividing cell with the capacity to differentiate.

“Mammal” as used herein refers to any member of the class Mammalia, including, without limitation, humans and nonhuman primates such as chimpanzees, and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be included within the scope of this term.

“Therapeutically effective amount” as used herein refers to that amount which is capable of achieving beneficial results in a patient in need of treatment. A therapeutically effective amount can be determined on an individual basis and will be based, at least in part, on consideration of the physiological characteristics of the mammal, the type of delivery system or therapeutic technique used and the time of administration relative to the progression of the disease.

“Treatment” and “treating,” as used herein refer to both therapeutic treatment and prophylactic or preventative measures, wherein the object is to prevent, slow down and/or lessen the disease, or repair the tissue even if the treatment is ultimately unsuccessful.

“Conditions” and “disease conditions,” as used herein may include, but are in no way limited to any form of cardiac disease, neurological deficit, bone injury, cartilage injury, joint injury, degenerative spinal disease, vascular disease, viral infection, and tissue damage.

AASPCs interact closely with preadipocytes during their transition to mature adipocytes. It appears that cellular cross-talk may be important in modulating the proliferation of preadipocyte precursors. For example, AASPCs are present in small numbers in tissue and in culture, and these cells do not proliferate in vitro until adipocytes and resident macrophages are co-cultured together (Seta, N. and M. Kuwana, Human circulating monocytes as multipotential progenitors. Keio J Med, 2007. 56(2): p. 41-7). The inventors believe that the resident macrophages in adipose tissue are a source of preadipocytes and eventually modulate the balance of preadipocytes to adipocytes. As described herein, direct cell to cell co-culture of adipocytes, macrophages, and AASPCs results in the robust proliferation of preadipocytes, due in part through the differentiation of macrophages to preadipocytes. Additionally, direct cell to cell contact of adipocytes and macrophages increases the proliferation of novel adult adipocyte stem/progenitor cells that link obesity and inflammation.

Obesity and inflammation are closely linked. Co-culturing of adipocytes with the resident macrophage fraction allows for cross-talk to occur between adipocytes, macrophages, and AASPCs. The result is an increase in preadipocyte proliferation and a large increase in adiponectin. Both adiponectin and preadipocytes have been shown to be inversely proportional to adipogenesis and obesity, although mechanisms behind this observation are unknown. In fact, the primary preadipocytes marker, DLK, was shown to have an inhibitory effect on adipocyte differentiation (Kim et al., Pref-1 (preadipocyte factor 1) activates the MEK/extracellular signal-regulated kinase pathway to inhibit adipocyte differentiation. Mol Cell Biol, 2007. 27(6): p. 2294-308). The inventors demonstrated herein that macrophages can differentiate to preadipocytes. This differentiation is increased with cross-talk, and cross-talk is dependent on close cell-to-cell interactions. Co-cultures using a membrane have had muted effects, and gap junctions such as connexin 43 have been shown to be required for adipogenesis (Yanagiya et al., Gap-junctional communication is required for mitotic clonal expansion during adipogenesis. Obesity (Silver Spring), 2007. 15(3): p. 572-82). Stem cells taken from adipose tissue have been shown to be hematopoietic in origin. Using the co-culturing methods described herein will allow for an enhanced proliferation of preadipocytes, cells that when isolated can be directed to differentiate down osteogenic, chondrogenic, adipogenic, myogenic, cardiomyogenic, neurogenic, angiogenic, or dendritic pathways. Preadipocyte proliferation can be linked to reduced adipogenesis. Targeting preadipocytes may allow for new treatments of obesity, inflammation, and related diseases such as diabetes and Polycystic Ovary Syndrome.

The results herein demonstrate that cross-talk between adipocytes, macrophages, and CD34(+) AASPCs occurs naturally in adipose tissue of obese individuals. This would also lead one to anticipate that the changes due to cross-talk that are seen in co-culture in vitro are observable, while perhaps more subtly, in the adipose tissue in vivo.

### Obesity and Inflammation

Adipose tissue, which acts as an active secretory organ, is composed of complex populations of cells that affect insulin sensitivity, reproductive and endocrine systems, immunity, and inflammation (Fantuzzi, G., Adipose tissue, adipokines, and inflammation. J Allergy Clin Immunol, 2005. 115(5): p. 911-9; quiz 920). There are numerous lines of evidence that link the clear and prominent role of inflammation in obesity. Currently the most complete way to study inflammations link to obesity in vitro is to stimulate adipocyte fat cells with the corresponding resident macrophages through direct cell to cell contact. Having first been demonstrated in cultures of stable adipocyte and macrophage murine cell lines, a definitive biological process was shown to occur during the cross-talk of these two cell populations (Mitchell et al., Immunophenotype of human adipose-derived cells: temporal changes in stromal-associated and stem cell-associated markers. Stem Cells, 2006. 24(2): p. 376-85). While the ultimate reason for the increased production of proinflammatory factors by adipose tissue in obesity has not been elucidated, cross-talk between lymphocytes and adipocytes leads to immunoregulation in the body and is responsible for the release of adipokines such as leptin, adiponectin, resistin, and visfatin, as well as cytokines such as TNF-a, IL-6, and MCP-1 (Fantuzzi, G., Adipose tissue, adipokines, and inflammation. J Allergy Clin Immunol, 2005. 115(5): p. 911-9; quiz 920).

### Co-Culture of Macrophages/AASPCs and Adipocytes

Approximately 10% of white adipose tissue is composed of CD14(+), CD31(+) macrophages. Macrophages in the white adipose tissue are bone marrow derived and arise from circulating monocytes that infiltrate and accumulate in the adipose tissue (Weisberg et al., Obesity is associated with macrophage accumulation in adipose tissue. J Clin Invest, 2003. 112(12): p. 1796-808). Although it is yet to be defined, there also exists a link between macrophages and diabetes. Macrophages have been shown to block insulin action in adipocytes (Lumeng et al., Macrophages block insulin action in adipocytes by altering expression of signaling and glucose transport proteins. Am J Physiol Endocrinol Metab, 2007. 292(1): p. E166-74), and in obese individuals, macrophages stimulated with proinflammatory cytokines lead to an increase in resistin, an insulin resistance gene and protein.

To measure the necessity of direct cell to cell contact between adipocytes and resident macrophages, experiments were performed with and without a mesh. In cultures using a mesh, the inventors observed a reduction in adiponectin secretion (data not shown). Proliferation of preadipocytes is lessened significantly while culturing with a mesh barrier. One possible reason for this muted cross-talk effect could be that the mesh prevents gap junction formation between the macrophages/AASPC fraction and the adipocyte fraction. This correlates to findings that conclude gap junction proteins such as connexin 43 are needed for mitotic clonal expansion during adipogenesis (Yanagiya et al., Gap-junctional communication is required for mitotic clonal expansion during adipogenesis. Obesity (Silver Spring), 2007. 15(3): p. 572-82). This again illustrates the necessity of cell to cell contact in co-culture. Cell to cell contact does not necessarily guarantee that successful cross-talk will occur. Monocytes taken from blood were isolated and allowed to reach equilibrium in culture for 24 hours before being co-cultured with adipocytes. The resulting culture had no preadipocytes or fibroblast-like cells, and the monocytes were unchanged (data not shown). Resident macrophages may be required for optimal co-culture conditions to be met.

Cross-talk between two different stable murine cell lines has many limitations, however, and does not react with the same vigor as cells isolated and cultured from one source. The inventors have been able to co-culture human adipocytes and resident macrophages in excised tissue from obese patients to show a novel pathway in which co-cultured cells can be driven to differentiate into several lineages in highly proliferative states within a matter of days. During the co-culture period, the inventors observed vast amounts of preadipocytes often becoming confluent 24 hours after cross-talk initiates. This cell proliferation correlates to a sharp increase in adiponectin, a hormone responsible for the regulation of metabolic processes such as glucose regulation and fatty acid catabolism, which is only secreted by adipose tissue, adipocytes and preadipocytes (Schaffler et al., Role of adipose tissue as an inflammatory organ in human diseases. Endocr Rev, 2006. 27(5): p. 449-67). Preadipocytes over-express secreted adiponectin before they begin adipocyte differentiation. Adiponectin levels were measured in the macrophage section of cultures with and without cross-talk, showing an increase in adiponectin of 18 fold during cross-talk (FIG. 4 D). These adiponectin levels indicate a great increase in preadipocytes. While produced by fat tissue, adiponectin levels are inversely proportional to BMI, and this relationship is still not fully understood.

It would appear that the key of this co-culture lies in the resident macrophage section. The macrophages themselves perform two main functions in regard to adipocytes. Macrophages phagocytize the adipocyte debris created as hypoxia inflicts damage on newly formed adipocytes that proliferate at extremely high rates, as seen especially in cases of obesity with inflammation. Another function of macrophages is to produce proinflammatory cytokines that increase adipogenesis and insulin resistance. (Simmons et al., Vascular cell adhesion molecule-1 expressed by bone marrow stromal cells mediates the binding of hematopoietic progenitor cells. Blood, 1992. 80(2): p. 388-95; Bouloumie et al., Role of macrophage tissue infiltration in metabolic diseases. Curr Opin Clin Nutr Metab Care, 2005. 8(4): p. 347-54)

### Macrophages and Preadipocyte Characterization

Resident macrophages mainly derive from bone marrow, though recent discoveries have shown macrophages to also have progenitor cells in the tissue in which it infiltrates (Cousin et al., A role for preadipocytes as macrophage-like cells. Faseb J, 1999. 13(2): p. 305-12). Adipose tissue has been shown to contain stem cells that have a high plasticity, with the ability to differentiate down several mesenchymal lineages including adipocytes, smooth muscle cells, dendritic cells, osteoblasts, myocardiocytes and even neural cells (Fraser et al., Fat tissue: an underappreciated source of stem cells for biotechnology. Trends Biotechnol, 2006. 24(4): p. 150-4; Moseley et al., Adipose-derived stem and progenitor cells as fillers in plastic and reconstructive surgery. Plast Reconstr Surg, 2006. 118(3 Suppl): p. 121S-128S; Charriere et al., Macrophage characteristics of stem cells revealed by transcriptome profiling. Exp Cell Res, 2006. 312(17): p. 3205-14).

Resident macrophages present in the stromal-vascular fraction of the excised abdominal adipose tissue are monocyte derived (Sengenes et al., The role of endothelial cells in inflamed adipose tissue. J Intern Med, 2007. 262(4): p. 415-21). Monocytes are thought to fall into one of two groups, short-lived recruited monocytes or a monocyte subset that constitutively homes tissues (Geissmann et al., Blood monocytes consist of two principal subsets with distinct migratory properties. Immunity, 2003. 19(1): p. 71-82). In recent unpublished FACS data, the inventors have observed preadipocytes forming from multiple origins. These preadipocytes, which were S-100 and DLK positive, formed from both CD14(+) and CD14(−) precursors, and within the CD14(+) group, there exists preadipocytes that are CD34(+) and CD34(−). This would imply a complex population of cells, with preadipocytes deriving from multiple sources.

Most preadipocytes observed were CD14(+) to varying degrees. As a cell became more differentiated it would have less CD14(+) staining of its membrane (FIG. 7). CD14 is not found on mature adipocytes (Festy et al., Surface protein expression between human adipose tissue-derived stromal cells and mature adipocytes. Histochem Cell Biol, 2005. 124(2): p. 113-21), indicating that preadipocytes that were CD14(−) were far enough along in their differentiation that they had lost CD14 label, or these cells dedifferentiated from adipocytes. The vast majority of cells derived from co-culture were CD14(+), however, which excludes them from being dendritic cells. Macrophages and dendritic cells share the macrophage/monocyte marker CD68, and adipocytes, preadipocytes and dendritic cells are both positive for S-100, but only macrophages are CD14(+) (Vakkila et al., A basis for distinguishing cultured dendritic cells and macrophages in cytospins and fixed sections. Pediatr Dev Pathol, 2005. 8(1): p. 43-51).

Monocyte-Derived Multipotential Cells (MOMC) are described as being CD14(+) and CD34(+) progenitor cells that can be derived from circulating CD14(+) positive monocytes and driven down several different cell differentiation pathways (Seta, N. and M. Kuwana, Human circulating monocytes as multipotential progenitors. Keio J Med, 2007. 56(2): p. 41-7). MOMCs appear to be the result of bone marrow derived cells, and these can differentiate to fibroblast-like cells. This closely mirrors macrophage to preadipocyte differentiation that the inventors observed. The CD34 on the MOMCs can also be found on the cell surface of some mature adipocytes (Festy et al., Surface protein expression between human adipose tissue-derived stromal cells and mature adipocytes. Histochem Cell Biol, 2005. 124(2): p. 113-21). In fact, CD34(+) cells compose 10% of the total white stromal-vascular fraction (Prunet-Marcassus et al., From heterogeneity to plasticity in adipose tissues: site-specific differences. Exp Cell Res, 2006. 312(6): p. 727-36). If there are preadipocytes that are derived from macrophages with monocyte and bone marrow progenitors, the CD34(+) mature adipocyte may be explained.

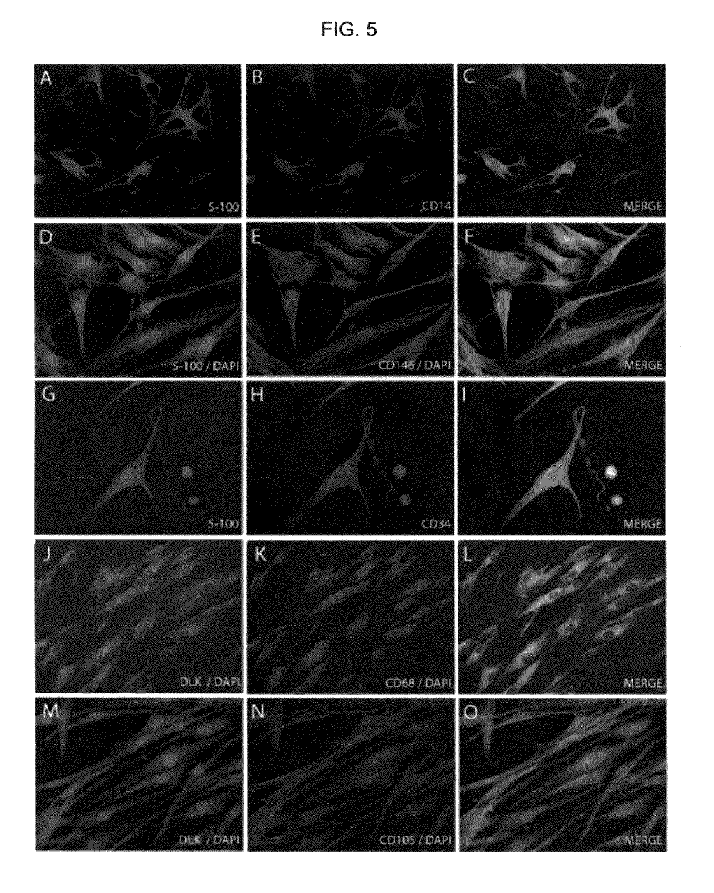

CD146, an epithelial cell and epithelial stem cell marker, and CD105, a mesenchymal stem cell marker, have both been found to be expressed in MOMCs. In preadipocytes derived from co-cultures, CD146 and CD105 have both been observed (FIG. 5 E, N). MOMCs derived from CD14(+) monocytes are CD34(+) before taking on a more endothelial CD146(+) phenotype. Cells from the stromal-vascular fraction from adipose tissue are comparable to bone marrow mononuclear cells in their proangiogenic potential (Planat-Benard et al., Plasticity of human adipose lineage cells toward endothelial cells: physiological and therapeutic perspectives. Circulation, 2004. 109(5): p. 656-63). It has been postulated that both adipocytes and endothelial cells have a common progenitor (Planat-Benard et al., Plasticity of human adipose lineage cells toward endothelial cells: physiological and therapeutic perspectives. Circulation, 2004. 109(5): p. 656-63). Preadipocytes, described as dedifferentiated adipocytes, were reported as quickly acquiring an endothelial phenotype and becoming CD146(+) as the cells promoted neovascularization.

Co-culture of macrophages and adipocytes lead to preadipocyte proliferation and CD105 labeled these resulting preadipocytes strongly. FIG. 5 shows the merge of CD105 positive preadipocytes with DLK, Preadipocyte Factor-1. While this labeling suggests mesenchymal origins, CD105(+) label is often found in bone marrow derived progenitor cells. Progenitor cells derived from bone marrow can express CD105, which has been isolated from adipose tissue, bone marrow, and fetal lung. CD105(+) cells can be driven to differentiate to osteoblasts, chondrocytes, adipocytes, smooth muscle, or skeletal muscle (Lakshmipathy, U. and C. Verfaillie, Stem cell plasticity. Blood Rev, 2005. 19(1): p. 29-38).

### Macrophage to Preadipocyte Differentiation

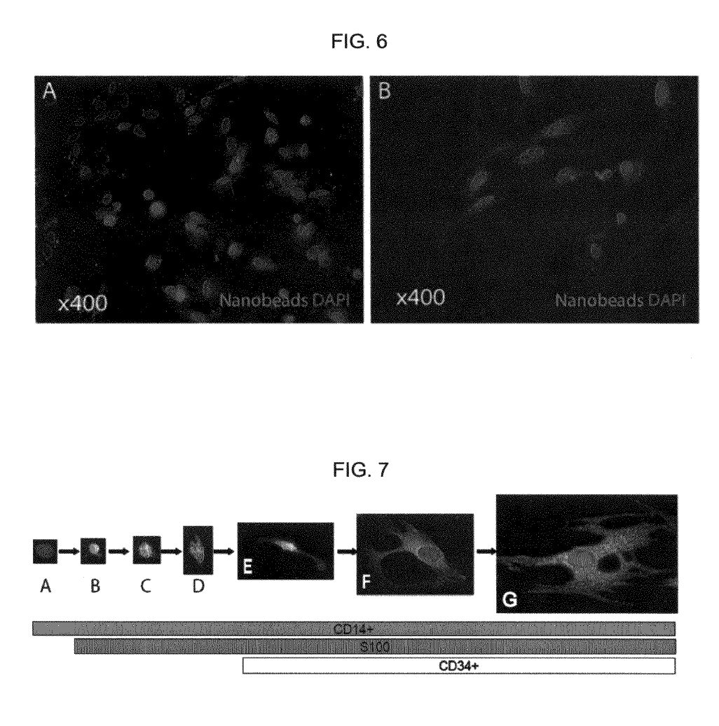

The macrophage fraction was incubated with anti-human CD14 nanobeads prior to co-culture and temperature modulation induced nanobead phagocytosis of the nanobeads. A Ficoll followed the incubation, so as to remove any unbound nanobeads, and while this density gradient resulted in an additional loss of cells from the macrophage fraction, it was very effective in removing the unbound nanobeads. Nearly all the macrophages took the beads into their cytoplasm (FIG. 6A). The resulting co-culture with adipocytes yielded preadipocytes (labeled for S100 and DLK; not shown) that contained nanobeads in a relatively similar ratio as macrophages containing nanobeads.

The resident macrophage fraction does not only include macrophages, as it also contains adult adipose stem/progenitor cells. Any of these cells are present as macrophages differentiate to preadipocytes when induced by cross-talk with adipocytes, as shown by the nanobead experiment. However, with the majority of cells derived in the macrophage fraction being macrophages (CD14+), and with the majority of cells from the macrophage fraction incorporating the anti-human CD14 nanobeads, one can conclude that because nanobeads were found in the majority preadipocytes, the macrophages are the cells that have differentiated. The preadipocytes had nanobeads comparable to both the number of macrophages that took up the nanobeads and the number macrophages that are in the macrophage fraction.

Preadipocytes have been shown to differentiate to macrophages, dedifferentiate from adipocytes, and differentiate back to adipocytes. Macrophages and monocytes are routinely differentiated to CD68(+), S100(+), CD14(−) dendritic cells, and monocytes are closely linked with all recent discussions of circulating stem/progenitor cells. Bone marrow derived stem cells have been induced to differentiate into large multinucleated myofibers or skeletal muscle (Lakshmipathy, U. and C. Verfaillie, Stem cell plasticity. Blood Rev, 2005. 19(1): p. 29-38). As for macrophages and adipocytes, both have been found to express adipsin (complement factor D), the adipocyte differentiation-dependent serine protease gene (White et al., Human adipsin is identical to complement factor D and is expressed at high levels in adipose tissue. J Biol Chem, 1992. 267(13): p. 9210-3). It has been demonstrated that macrophages in co-culture developed an increase in accumulation of cytoplasmic lipid vacuoles. In direct co-culture with adipocytes, peritoneal macrophages changed morphology to one with an elongated appearance with long cellular extensions (Lumeng et al., Macrophages block insulin action in adipocytes by altering expression of signaling and glucose transport proteins. Am J Physiol Endocrinol Metab, 2007. 292(1): p. E166-74).

Microarray studies comparing macrophages and progenitor/stem cells show a strong correlation in endocytosis, vesicle trafficking, and actin remodeling. Interestingly, transcription profiles of ESCs and macrophages were clustered together, unlike differentiated ESCs (Charriere et al., Macrophage characteristics of stem cells revealed by transcriptome profiling. Exp Cell Res, 2006. 312(17): p. 3205-14). It was found that preadipocytes actually participate in phagocytic activity and cells that are more stem-like show a direct correlation to phagocytic ability. Approximately 95% of peritoneal macrophages, 45% of mesenchymal stem cells and 35% of preadipocytes displayed phagocytic behavior, as compared to only 1% of fibroblasts doing the same (Charriere et al., Macrophage characteristics of stem cells revealed by transcriptome profiling. Exp Cell Res, 2006. 312(17): p. 3205-14). While there is a large drop off in preadipocytes undergoing phagocytosis, this could be expected with a gradual change in cell behavior as differentiation moves to favor the preadipocytes. Alternatively, preadipocytes arise from several sources, and phagocytic activity may identify the preadipocytes with a macrophage progenitor.

With obesity being responsible for increased inflammation and macrophage recruitment and infiltration to adipocytes, one might expect for lean or malnourished individuals with little to no adipogenesis to display a malfunctioning inflammatory system, perhaps with impaired macrophage recruitment and infiltration. However, there is not a lowered immunity in the malnourished, such as patients with anorexia nervosa (Fantuzzi, G., Adipose tissue, adipokines, and inflammation. J Allergy Clin Immunol, 2005. 115(5): p. 911-9; quiz 920). This indicates that while adipocytes may require cross-talk with resident macrophages for increased adipokine release, macrophages are not necessarily dependent on adipocytes for recruitment, activation, or infiltration to a tissue in cases of inflammation.

### Adult Adipocyte Stem/Progenitor Cells (AASPCs)

The inventors have also identified a novel AASPC, previously undescribed, with the potential of plasticity. The inventors have measured this new AASPC's diameter to at times be less than 0.5 microns, with an average size of about 1.0-1.5 microns. This tiny cell can be found on preadipocytes in clusters after 24 hours of cross-talk between adipocytes and resident macrophages. The AASPC's emergence correlates to the onset of CD34 expression in culture. Before cross-talk, both resident macrophage and adipocyte cultures are negative for CD34. CD34 mediates the attachment of hematopoietic stem cells directly to stromal cells. The AASPCs float in culture, a trait of hematopoietic cells. However, they adhere tightly to preadipocytes that are attached to the dish.

Embryonic stem cells are capable of phagocytizing other cells, and the inventors have observed the AASPCs act in a similar manner. As AASPCs selectively attach to preadipocytes, they become CD34 positive and extremely S-100 positive. While not wishing to be bound by any particular theory, the inventors believe that the AASPCs can selectively choose the preadipocytes with which they wish to communicate and phagocytize. This observation suggests a new role of stem cells in which the cell clears debris of dead or dying cells as it clears a path for new cells to proliferate. AASPCs can choose to completely engulf a specific preadipocyte, while surrounding preadipocytes go about proliferating without interruption. AASPCs do not attach near the nucleus of the preadipocyte, and the nucleus of the devoured preadipocyte is left behind after phagocytosis. Macrophages devour entire cells, including the nucleus after receiving apoptotic signals that draw them to the targeted cell. This would suggest that AASPCs lack this factor, as they can perform phagocytosis but are not true macrophages.

### Mirror Systems and Surgical Applications

During macrophage differentiation to preadipocytes, the inventors observed chimeric cells CD14(+) and S-100(+). As the cell became more differentiated there is less CD14 and more S-100(+) (FIG. 7). One can likely find systems of cross-talk mirrored in other biological processes and disease states. For instance, it is known that monocytes are recruited to endothelial cells for vasculogenesis. Monocytes appear to be able to differentiate into an endothelial precursor population (Kuwana, et al., Endothelial differentiation potential of human monocyte-derived multipotential cells. Stem Cells, 2006. 24(12): p. 2733-43). Another example can be seen in a chimeric cell implicated in diseases such as scleroderma that is CD68(+), S-100(+), and CD14(−). These chimeric cells express less and less CD14 as they differentiate, but CD14(+) cells do make up 24% of the surrounding population (McNallan et al., Immunophenotyping of chimeric cells in localized scleroderma. Rheumatology (Oxford), 2007.46 (3): p. 398-402). A similar cell may be found in atherosclerotic plaques. It has long been thought that there is a circulating progenitor cell component to atherosclerosis. DC-SIGN, a C-type lectin expressed by dendritic cells, monocytes and macrophages, has been shown to be expressed in atherosclerotic plaques. DC-SIGN(+) cells were also co-labeled for CD14(+), CD68(+), and S-100(+) (Soilleux et al., Human atherosclerotic plaques express DC-SIGN, a novel protein found on dendritic cells and macrophages. J Pathol, 2002. 198(4): p. 511-6).

These cells also have the potential for surgical applications. Adipocytes are abundant and easily harvested cells, with patients being open to their removal and use and plastic surgeons already proficient in its extraction and manipulation. Surgery utilizing fat cells has the advantage of using the adipose tissue itself, which provides a scaffold on which preadipocytes and stem/progenitor cells can grow. Cross-talk would potentially help to enhance growth and proliferation of the preadipocytes and AASPCs. Also, the significant angiogenic potential of the AASPCs that comes with preadipocyte formation is another advantage of surgical applications (Moseley et al., Adipose-derived stem and progenitor cells as fillers in plastic and reconstructive surgery. Plast Reconstr Surg, 2006. 118(3 Suppl): p. 121S-128S).

Embodiments of the present invention are based on, inter alia, the inventors' discovery that new populations of adipocytes, preadipocytes and a novel population of adult adipocyte stem/progenitor cells (“AASPCs”) may be generated through co-culturing of adipocytes and resident tissue macrophages. The newly identified AASPCs have displayed phagocytotic ability of macrophages as well as markers commonly found in preadipocytes and adipocytes.

Accordingly, the present invention provides for a method of producing preadipocytes. The method comprises providing a population of adipocytes and a population of macrophages; and co-culturing the population of adipocytes and the population of macrophages to differentiate the macrophages into preadipocytes. In one embodiment, an AASPC is present in the population of macrophages.

The population of adipocytes and the population of macrophages may be isolated from adipose tissue. Thus, in one embodiment, the macrophages are resident adipose tissue macrophages. In a particular embodiment, the adipose tissue is human adipose tissue.

Co-culturing the population of adipocytes and the population of macrophages may comprise co-culturing for a period of about 24 hours. In a further embodiment, the method further comprises separating the population of adipocytes and the population of macrophages and separately culturing the population of macrophages, whereby the macrophages differentiate into preadipocytes. The preadipocytes may be extracted or isolated and allow to proliferate indefinitely in culture. As such, a purified and/or isolated population of preadipocytes is obtained.

The present invention also describes AASPCs. In a particular embodiment, the invention provides for a purified preparation of AASPCs. In a one embodiment, the AASPCs are about 1.0 to about 1.5 microns. In another embodiment, the AASPCs are less than 0.5 microns. In another embodiment, the AASPCs are about 0.5 to about 1.0 microns. In another embodiment, the AASPCs are more than 1.5 microns.

In a further embodiment, the AASPCs are capable of phagocytizing other cells; particularly preadipocytes.

In another embodiment, the AASPCs express CD34, i.e., they are CD34+. In another embodiment, the AASPCs express S-100, i.e., they are S-100+. In another embodiment, the AASPCs express CD34 and S-100. In a particular embodiment, the AASPCs express S-100 at a much higher level than preadipocytes.

The present invention also provides for a method of increasing the proliferation of AASPCs. The method comprises providing a population of adipocytes and a population of macrophages comprising an AASPC; and co-culturing the population of adipocytes and the population of macrophages, whereby the AASPC proliferates in the co-culture conditions.

The macrophages may be resident tissue macrophages. In one embodiment, the population of adipocytes and the population of macrophages are isolated from adipose tissue. In a particular embodiment, the adipose tissue is human adipose tissue.

Co-culturing the population of adipocytes and the population of macrophages may comprise co-culturing for a period of about 24 hours. In a further embodiment, the method further comprises separating the population of adipocytes and the population of macrophages and separately culturing the population of macrophages, whereby the proliferation rate of the AASPCs is increased. The AASPCs may be extracted and/or isolated and allowed to proliferate indefinitely in culture. As such, a purified and/or isolated population of AASPCs is obtained.

The present invention also provides for a method of producing adipocytes. The method comprises providing an AASPC; culturing the AASPC in a culture condition that induces the AASPC to differentiate into a preadipocyte; and culturing the preadipocyte in a culture condition that induces the preadipocyte to transform into an adipocyte. The adipocytes may be extracted or isolated and thus a purified and/or isolated population of adipocytes is obtained.

In an alternative embodiment, the method comprises providing a population of adipocytes and a population of macrophages wherein an AASPC is present in the population of macrophages; and co-culturing the population of adipocytes and the population of macrophages, whereby the AASPC proliferates in the co-culture conditions and preadipocytes and adipocytes are produced in the co-culture conditions.

The macrophages may be resident tissue macrophages. In one embodiment, the population of adipocytes and the population of macrophages are isolated from adipose tissue. In a particular embodiment, the adipose tissue is human adipose tissue.

Co-culturing the population of adipocytes and the population of macrophages may comprise co-culturing for a period of about 24 hours. In a further embodiment, the method further comprises separating the population of adipocytes and the population of macrophages and separately culturing the population of adipocytes and the population of macrophages, whereby the AASPC proliferates and preadipocytes and adipocytes are produced in the co-culture conditions. The adipocytes may be extracted or isolated and thus a purified and/or isolated population of adipocytes is obtained.

The present invention also provides for methods of differentiating AASPCs into different cell types; for example, smooth muscle cells, cardiomyocytes, myocytes, osteoblasts, neural cells, hematopoietic cells, preadipocytes, and adipocytes. The method comprises providing an AASPC and culturing the AASPC in a culture condition that induces the AASPC to differentiate into the particular cell type.

The differentiation of these AASPCs may treat many diseases or conditions including cardiac disease, neurological deficits, bone, cartilage, and joint injuries, degenerative spinal disease, and vascular diseases.

In orthopedics, AASPCs produced and isolated after direct contact of isolated adipocytes and macrophages in the presence of one or more AASPCs could be differentiated into cartilage cells. The rate is significant so that enough cells can be generated fast enough and in large numbers to make this application practical. These AASPCs may be utilized to repair significant bone defects. In cardiology, AASPCs could be involved in the treatment of cardiac injury following acute myocardial infarction. Recently it was published that adipose tissue-derived stem cells enhance cardiac function following surgically induced myocardial infarction. Therefore, AASPCs also have the potential to improve left ventricular function after an acute myocardial infarction. In breast cancer, adult adipose-derived stem cells are being researched for use in breast reconstruction cancer therapy. By utilizing the patient's own adipocyte stem cells, reconstruction of their breast after partial mastectomy may become a viable option. Therefore the addition of AASPCs to the patient's own breast adipose tissue may better maintain the volume of the reconstructed tissue. In plastic surgery, AASPCs may be used in the augmentation of soft-tissue space fillers in plastic and reconstructive surgery. Also, they could provide a source of stem cells for fat grafting. In HIV and other viral infections, AASPCs have the potential to manipulate the immune system. Transfection of AASPCs or macrophages (before co-culture with adipocytes) with antiviral genes may be used for the treatment of many viral infections including HIV. The methods of the present invention generate AASPCs, which proliferate at a very high rate. Therefore, AASPCs may be the most efficient cells utilized for the treatments described above.

The present invention also provides for a method of using AASPCs. In one embodiment, the AASPCs may be used in reconstructive surgery. In another embodiment, the AASPCs may be used for tissue engineering. In another embodiment, the AASPCs may be used for in situ tissue regeneration. In one embodiment, the tissue regeneration occurs in a mammal. In a particular embodiment, the tissue regeneration occurs in human.

The use of adipose tissue, as a source of stem cells, is a low cost, high volume alternative to other stem cell sources. This allows patients to be treated using their own stem cells. This is an important breakthrough for adult stem cell therapies. The inventors' data demonstrate that these AASPCs appear to be hematopoietic stem cells. AASPCs produced after co-culturing of adipocytes and macrophages can thus be used in vivo.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

### Subjects

Adipose tissue was obtained from female patients undergoing abdominoplasty. All patients were premenopausal, non-diabetic, and none had been on any hormonal treatment, including oral contraceptives. Once the abdominal adipose tissue had been excised, the tissue was placed in buffer (Hepes/Salts, 4% BSA, 2 mM pyruvate, pH 7.4, at room temperature (RT). A small amount of tissue was subjected to immunofluorescence while the remainder was used for adipocyte and macrophage isolation.

### Example 2

### Isolation of Adipocytes and Resident Macrophages/AASPCs from Human Adipose Tissue

Tissue was finely minced and treated with collagenase (Worthington, Bioch. Corp., Lakewood, N.J.) for 60 min at 37° C., in the transport buffer. The cell suspension was then filtered through a pre-moistened 400-micron nylon mesh (Small Parts Inc, Miami Lakes, Fla.) and centrifuged for 2 min at 50×g at RT. The remaining cells in suspension (adipocytes) were then washed twice and diluted in adipocyte culture medium (DMEM 1% BSA, 3% FCS, Penicillin/Streptomycin). The stromal-vascular fraction, containing various cell components including resident macrophages and AASPCs, was resuspended in 10 ml of PBS. The resuspension was slowly layered on top of a Ficoll density gradient solution (Lymphoprep, Greiner Bio-one, Longwood, Fla.) and then centrifuged at 300×g for 30 mins. at 4° C. After centrifugation, the interface containing the macrophage fraction (i.e., resident macrophages and CD34(+) AASPCs) was removed and washed with 5 ml of PBS at RT. The cell pellet was resuspended in macrophage culture medium (RPMI medium supplemented with 10% FCS, 100 U/ml penicillin, 100 μg/ml streptomycin, 2 mM L-glutamine, 1% NEAA, 1% sodium pyruvate, and 10 ng/ml GMSF).

### Example 3

### Co-Culture of Adipocytes and the Macrophage Fraction

Cells from the macrophage fraction (10⁶/ml) and isolated adipocytes (10⁶/ml) were allowed to equilibrate separately overnight in their corresponding cell culture media (see above). Cover slips were placed in each well for immunofluorescence studies. Twenty-four hours later, the adipocytes were added to the wells containing the macrophage fraction at an approximate 1:1 ratio. The pooled cells were co-cultured for 24 hours at 37° C. in 5% CO₂. At the end of the incubation period the adipocytes were transferred by pipette and placed in another well containing 1 ml of adipocyte culture medium; the remaining macrophage fraction was washed twice with 1 ml PBS, and cultured in fresh macrophage culture medium. Both groups of cells were cultured separately for an additional 24 hours. At the completion of the post-co-culture incubation period coverslips were collected for immunofluorescence (see below). Culture media were also collected for determination of adiponectin levels (see below) at each stage of the culture process (after 24 hours of pre-co-culture incubation, after 24 hours of co-culturing and after 48 hours of post-co-culture incubation). Adipocytes and the macrophage fraction were cultured for 3 days without exposing them to co-culturing.

### Example 4

### Adiponectin Levels

The concentration of adiponectin (pg/ml) in the culture medium was determined in duplicate using a solid phase sandwich assay using enzyme-linked immunosorbent assay (ELISA; Dousset R&D System, Minneapolis, Minn.). Sample concentrations were determined using an ELISA Reader (Sunrise, T-can, Molecular Devices Vmax kinetic microplate reader, Sunnyvale, Calif.). Log transformation of the adiponectin levels was used in order to use parametric methods of analysis. Analysis between the log-transformed means of untreated samples and samples subjected to co-culturing was determined using a Student's t test.

### Example 5

### Immunofluorescence

Cells on the cover slips were fixed with 4% paraformaldehyde, washed with PBS, treated with 0.2% Triton-X-100, and then blocked with 10% goat serum in a 1% BSA solution. The primary antibody was incubated with the sample overnight at 4° C. The following day the slides were washed with PBS followed by a 1 hour treatment with the corresponding secondary antibodies and Alexa Fluor 568 and 488 (Invitrogen, Carlsbad, Calif.). After treatment, the slides were washed with PBS. Cover slips were then mounted on slides with mounting medium containing DAPI nuclei stain (Vectashield, Vector, Burlingame, Calif.). Whole tissue mounts were manually cut from excised tissue before being placed in a well and treated identically to cultured cells. Additional primary antibody staining used included: i) S-100, a selective marker of preadipocytes and newly formed adipocytes (Sigma Aldrich, St Louis, Mo.); ii) DLK, or Preadipocyte Factor-1, an exclusive preadipocyte marker (Santa Cruz, Santa Cruz, Calif.); iii) CD14, a macrophage/monocyte specific marker (Biosource, Camarillo, Calif.); iv) CD68, a macrophage, monocyte, and dendritic cell marker (BD Biosciences, San Diego, Calif.); v) CD34, an adult hematopoietic stem cell marker (Zymed, San Francisco, Calif.); vi) CD105, a mesenchymal stem cell marker (BD Biosciences, San Diego, Calif.); and vii) CD146 or MeI-CAM, an endothelial and endothelial stem cell marker (Santa Cruz Biotechnology, Santa Cruz, Calif.). Oil Red O staining was performed along with nuclear DAPI labeling to distinguish adipocytes from free lipid. Untreated adipocytes from culture were fixed in 4% paraformaldehyde, and rinsed twice for 10 minutes in PBS. Cells were stained with 0.35% Oil Red 0 (Sigma Aldrich, St. Louis, Mo.), in a 3:2 isopropanol/water solution for 10 minutes and rinsed in PBS.

### Example 6

### Nanobead Incubation

10⁷ cells from the macrophage fraction were incubated with 10 μl anti-human CD14 nanobeads, 200-300 nm in diameter (BD Biosciences, San Diego Calif.) for 30 minutes at 8° C. in 90 μl PBS, 0.5% BSA, 2 mM EDTA, and 0.09% sodium azide. After incubation, the mixture was layered over a Ficoll density gradient as described above to separate any unbound or non-phagocytized nanobeads from the macrophages. The incubated macrophage fraction was then plated for 24 hours and co-cultured as described above.

### Example 7

### Immunofluorescence of Adipose Tissue

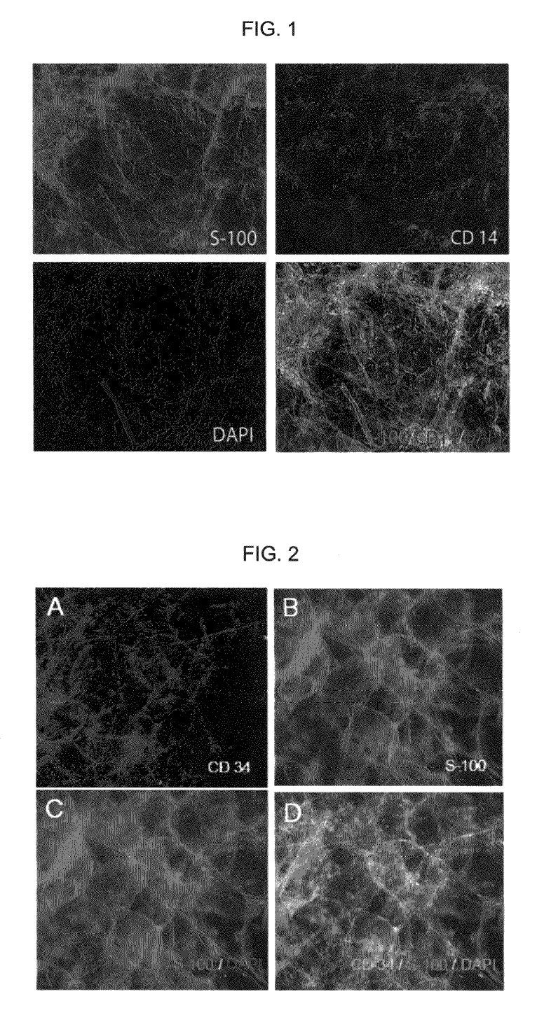

The presence of infiltrating macrophages in adipose tissue excised from an obese patient was determined by immunofluorescence. The tissue was treated and labeled as described above. As expected, the tissue was positive for S-100, a marker found in preadipocytes and recently matured adipocytes, indicating the presence of both preadipocytes and adipocytes, while displaying the cell's general morphology (FIG. 1A). Cells were also positive for CD14, clearly indicating the presence of infiltrating macrophages in the adipose tissue (FIG. 1B). Staining of the nucleus by DAPI is also observed (FIG. 1C). Areas of the merged image that appear yellow are points of macrophage/adipocyte interaction or overlap (FIG. 1D). To demonstrate the presence of AASPCs, adipose tissue was labeled with CD34, a marker for hematopoietic adult stem cells. Immunofluorescent CD34(+) cells were present in the adipose tissue (FIG. 2A). These cells were also S-100(+) (FIG. 2B) and stained positive for nuclei with DAPI labeling (FIG. 2C). Cells labeled with CD34/S-100/DAPI suggest an overlap between the phenotypes of many CD34(+) progenitors and adipocytes (FIG. 2D).

### Example 8

### Co-Culture Between Adipocytes, Macrophages, and AASPCs

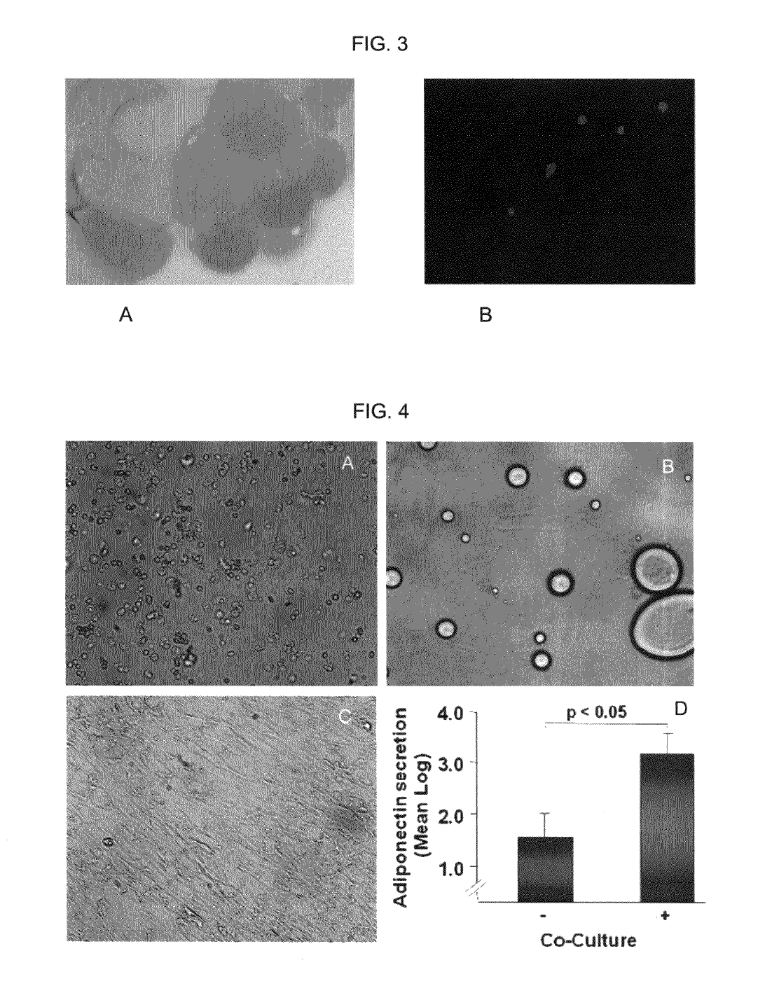

In vitro co-culturing of isolated adipocytes and the macrophages/AASPCs fraction results in the formation and proliferation of preadipocytes. The isolated adipocytes were characterized using Oil Red 0 and DAPI labeling (FIG. 3A). Isolated untreated macrophages can remain in culture for days without a significant change in cell population or morphology, and adhere tightly to the bottom of the culture wells, allowing for detailed immunofluorescence; the macrophage fraction expresses CD14 (FIG. 3 B) and CD68 (data not shown) before co-culturing, both classic macrophage markers.

The isolated adipocytes and macrophage/AASPCs cells were cultured separately for 24 hours to allow the cells to reach equilibrium and then were co-cultured for an additional 24 hours. Finally, the cells were separated and cultured for another 24 hours. Cells from the macrophage fraction that were plated and allowed 3 days of growth, but never underwent co-culture, experienced only a slight increase in preadipocyte proliferation, as demonstrated by immunofluorescence (FIG. 4 A). However, the macrophage fraction that was co-cultured for 24 hours with adipocytes had a significant increase in the proliferation of preadipocytes, often achieving confluency in the cell culture dish (FIG. 4 C). The macrophage fraction appears to require only nominal cross-talk for enhanced preadipocyte growth to occur; in fact, with a co-culture period of only two hours significant proliferation in preadipocytes is observed (data not shown). Alternatively, the adipocyte fraction, replated after co-culturing and cultured for an additional 24 hours, demonstrated only a small amount of preadipocyte formation 24 hours after cross-talk had completed (FIG. 4 B).

### Example 9

### Adiponectin Levels

Adiponectin is an adipokine exclusively of adipocyte origin, with levels of the hormone inversely proportionate to patient's BMI. Adiponectin levels were measured in the medium of the macrophage fraction at each stage of the co-culture. A comparison between the adiponectin levels in the media from the macrophage fraction with and without co-culture, 96 hours after initial plating, illustrates the stark difference achieved through cross-talk. Using mean log-transformed data, co-culture resulted in an increased secretion of adiponectin hormone of 173% (FIG. 4D) or 17-fold greater than that of untreated macrophages. The marked rise in adiponectin levels secreted into the media reflects the increased preadipocyte proliferation.

### Example 10

### Characterization Of Preadipocytes by Immunofluorescence

The preadipocytes that resulted from macrophage and adipocyte cross-talk were found to express a wide array of markers. As expected, all preadipocytes were S-100 (FIGS. 5 A, D, G) and DLK positive (FIGS. 5 J, M), which along with morphology, are indicative of the preadipocyte phenotype; DLK is absent in adipocytes and is found exclusively in preadipocytes. Interestingly, the preadipocytes obtained following co-culture were CD14(+), a marker that identifies macrophages/monocytes (FIGS. 5 B, C). Smaller preadipocytes appeared to be more strongly CD14(+), while the cell label dissipates in larger more differentiated preadipocytes. Preadipocytes obtained after co-culture were also positive for CD146, an endothelial cell and endothelial stem cell marker (FIGS. 5 E, F), CD34 (FIGS. 5 H, I), CD68(+) (FIGS. 5 K, L), and CD105 (FIG. 5 N). These cells were also co-labeled with DLK (FIG. 5 O), suggesting that preadipocytes arising from co-culture may have the ability to differentiate down mesenchymal pathways.

### Example 11

### Macrophage Differentiation to Preadipocytes

Preadipocyte formation could be attributed to: (i) differentiation of macrophages present in the stromal vascular fraction; (ii) differentiation of AASPCs also present in the stromal vascular fraction; and/or (iii) differentiation of macrophages that are bound to adipocytes or free floating during medium transfer. Immunofluorescence studies were performed to determine if macrophage to preadipocyte differentiation occurred, or if the preadipocytes were already present in culture and were merely activated by the co-culture induced cross-talk.

To prove conclusively that a subset of cells from the macrophage fraction was responsible for the expansive preadipocyte proliferation observed after co-culture, the inventors incubated macrophages with anti-human CD14 nanobeads. Nearly all macrophages incorporated beads into their cytoplasm before co-culture (FIG. 6A). After co-culture between the nanobeads-treated macrophage fraction and the untreated adipocytes, the majority of preadipocytes generated contained CD14 (+) nanobeads within their cytoplasm (FIG. 6B). The resulting preadipocytes were positive for S-100 and DLK (data not shown).

### Example 12

### Adult Adipocyte Stem/Progenitor Cells (AASPCs)

Immunofluorescence of adipose tissue found CD14(+) resident macrophages to have thoroughly infiltrated adipose tissue (not pictured), showing cross-talk in vivo. Adipose tissue was also CD34(+) displaying significant expression of the hematopoietic stem cell marker. Tissue sectioning of adipocytes excised from patients demonstrates the extent of naturally occurring cross-talk.

In vitro adipocytes and resident macrophages were co-cultured for 24 hours and immunofluorescence imaging was preformed and tiny AASPCs were found throughout the culture, ranging from 0.5 to 1.5 microns in diameter. These cells were in co-cultures that had undergone at least 2 hours of cross-talk, with high numbers attaching to preadipocytes after 24 hours of co-culturing.

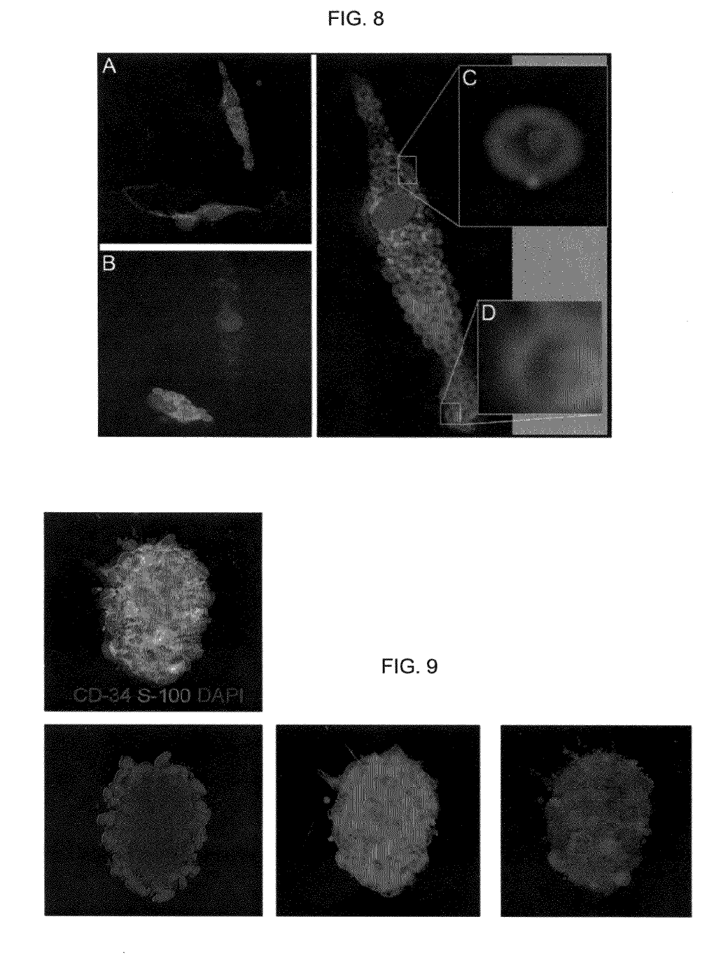

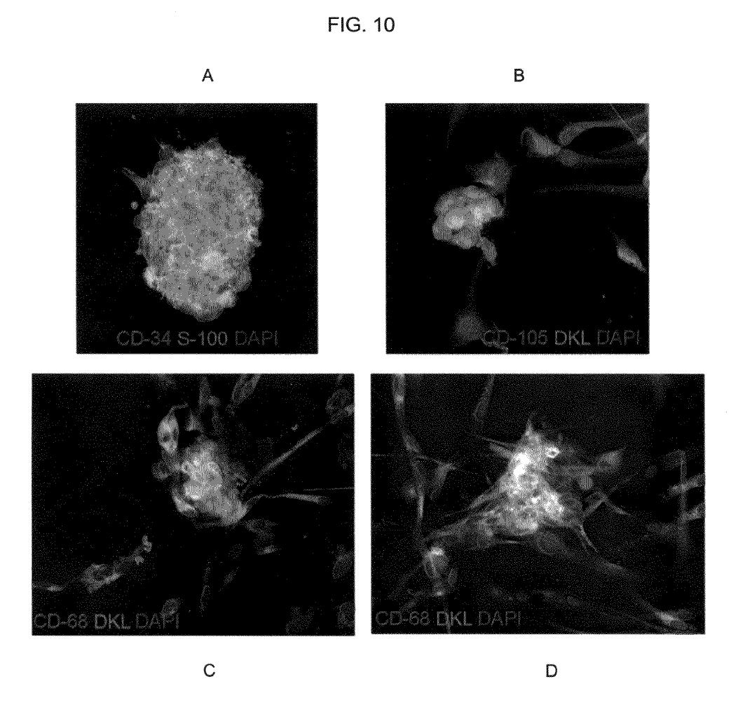

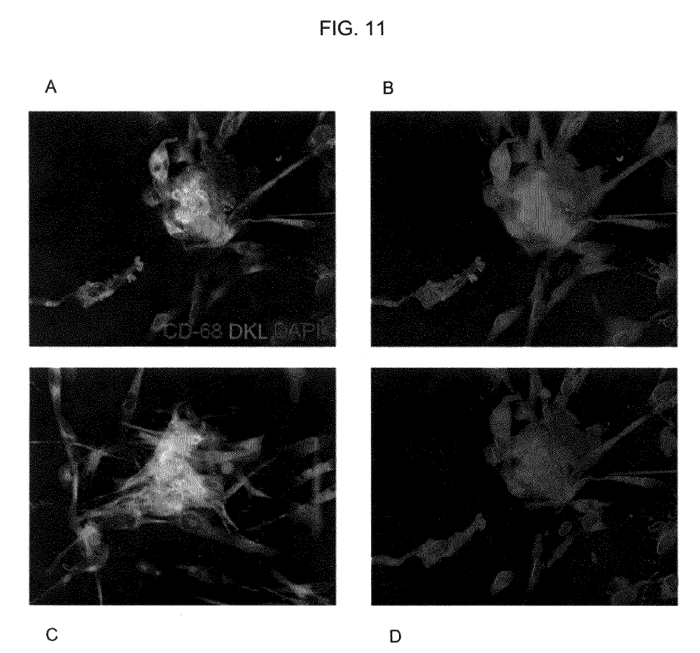

AASPCs were CD34(+) (FIG. 8D). CD34 is an inter-cellular adhesion molecule and cell surface glycoprotein and is believed to possibly mediate the attachment of hematopoietic stem cells directly to stromal cells. This would correlate to findings showing CD34(+) staining between adipocytes and macrophages as well as positive staining expressed at adhesion points of AASPCs with preadipocytes. Interestingly, these AASPCs firmly attach to approximately a third of the preadipocytes. This attachment is apparently selective since seemingly identical preadipocytes are completely engulfed in AASPCs or left untouched. FIG. 8A shows the bottom preadipocyte undergoing mitosis left undisturbed by AASPCs while the preadipocyte directly above is engulfed by AASPCs, leaving only the DAPI positive nucleus uncovered. While both preadipocytes and AASPCs are S-100(+) and CD34(+) (FIGS. 8A and 8B), preadipocytes show positive S-100 staining with fluorescent light exposure of 1-2 seconds, with AASPCs displaying positive results with only a 1 millisecond exposure time. While not wishing to be bound by any particular theory, the inventors believe that the great discrepancy in exposure strengths suggests that the AASPCs express S-100 at much higher levels, or more readily allow S-100 antibody to bind their domain.

Individual AASPCs were originally identified through the clear DAPI staining of their small nuclei (FIG. 8C). CD34 expression was found at cell to cell interaction points of AASPCs and preadipocytes. Confocal imaging of immunofluorescence was preformed to more closely study this interaction (FIG. 8B). Another interesting occurrence was the apparent phagocytosis of preadipocytes by the much smaller AASPCs. The preadipocytes were clearly seen at different stages of being devoured. However, unlike macrophages that phagocytize entire cells, AASPCs leave the nucleus of preadipocytes untouched (see top cell in FIG. 8A), often times resulting in preadipocyte nuclei that are left bare in culture (see bottom cell in FIG. 8B).

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.).

What is claimed is:

1. A method of producing preadipocytes, comprising:
providing a population of macrophages and a population of adipocytes; and
co-culturing the population of macrophages and the population of adipocytes to differentiate at least one macrophage into a preadipocyte.

2. The method of claim 1, wherein the population of macrophages is isolated from adipose tissue.

3. The method of claim 1, wherein co-culturing comprises co-culturing the population of macrophages and the population of adipocytes for about 24 hours.

4. The method of claim 3, further comprising separating the population of macrophages and the population of adipocytes and further culturing the population of macrophages.

5. A population of preadipocytes produced by the method of claim 1.

6. The population of preadipocytes of claim 5, wherein the population of macrophages is isolated from adipose tissue.

7. The population of preadipocytes of claim 5, wherein co-culturing comprises co-culturing the population of macrophages and the population of adipocytes for about 24 hours.

8. The population of preadipocytes of claim 7, wherein the method further comprises separating the population of macrophages and the population of adipocytes and further culturing the population of macrophages.

9. The population of preadipocytes of claim 5, wherein the method further comprises purifying the population of preadipocytes to generate a purified population of preadipocytes.

10. A method of increasing the proliferative rate of adult adipose stem/progenitor cells (“AASPCs”), comprising:
providing a population of adipocytes and a population of macrophages comprising an AASPC; and
co-culturing the population of adipocytes and the population of macrophages, whereby the proliferative rate of the AASPC increases.

11. The method of claim 12, wherein the population of macrophages is isolated from adipose tissue.

12. The method of claim 12, wherein co-culturing comprises co-culturing the population of adipocytes and the population of macrophages comprising the AASPC for about 24 hours.

13. A population of adult adipose stem/progenitor cells (“AASPCs”) produced by the method of claim 10.

14. The population of AASPCs of claim 13, wherein the population of macrophages is isolated from adipose tissue.

15. The population of AASPCs of claim 13, wherein co-culturing comprises co-culturing the population of adipocytes and the population of macrophages comprising the AASPC for about 24 hours.

16. A method of producing preadipocytes, comprising:
providing a population of adult adipose stem/progenitor cells (“AASPCs”) produced by:
providing a population of adipocytes and a population of macrophages comprising an AASPC, and
co-culturing the population of adipocytes and the population of macrophages to increase the proliferative rate of the AASPC to produce the population of AASPCs; and
differentiating the population of AASPCs into preadipocytes.

17. The method of claim 16, wherein the population of macrophages is isolated from adipose tissue.

18. The method of claim 16, wherein co-culturing comprises co-culturing the population of adipocytes and the population of macrophages comprising the AASPC for about 24 hours.

19. A method of using adult adipose stem/progenitor cells (“AASPCs”) to treat a condition in a subject in need thereof, comprising:
providing a quantity of AASPCs;
administering a therapeutically effective amount of the quantity of AASPCs to the subject to treat the condition.

20. The method of claim 19, wherein the condition is selected from the group consisting of cardiac disease, neurological deficit, bone injury, cartilage injury, joint injury, degenerative spinal disease, vascular disease, viral infection, tissue damage and combinations thereof.

21. The method of claim 20, wherein the condition is tissue damage and the quantity of AASPCs promotes tissue regeneration.
